(19) United States
(12) Patent Application Publication
(10) Pub. No.: US 2025/0241552 A1
(43) Pub. Date: Jul. 31, 2025

(54) FABRIC MODULE CAPABLE OF DETECTING A PHYSIOLOGICAL CONDITION BY UTILIZING A RADIO FREQUENCY SIGNAL AND DETECTION METHOD FOR USING THE SAME

(71) Applicant: SIL RADAR TECHNOLOGY INC., New Taipei City (TW)

(72) Inventors: I-Chu LIN, New Taipei City (TW); Shao-An SU, New Taipei City (TW)

(21) Appl. No.: 18/671,365

(22) Filed: May 22, 2024

(30) Foreign Application Priority Data
Jan. 31, 2024 (TW) 113103766

(51) Int. Cl. A61B 5/05; A61B 5/00

(52) U.S. Cl. CPC A61B 5/05; A61B 5/6804; A61B 5/7225; A61B 5/7228; A61B 5/7257; A61B 2562/164

(57) ABSTRACT

A fabric module including a weaving body, a conductor group, a signal processing unit, and a sensing unit is disclosed. The weaving body is directly or indirectly contacted with a user. The conductor group includes at least two conductors spaced apart from each other and is attached to the weaving body. The signal processing unit emits a radio frequency signal to the conductor group and receives a feedback signal generated by the conductor group based on the radio frequency signal. The signal processing unit correspondingly generates a time domain periodically signal wave diagram and a frequency domain periodically signal wave diagram based on the feedback signal and detects the physiological condition of the user based on at least one of the time domain signal wave diagram and frequency domain periodically signal wave diagram.

### BACKGROUND OF THE DISCLOSURE

### Technical Field

The present disclosure relates to a fabric module, which is particularly to a fabric module capable of detecting a physiological condition, and a detection method utilized by the fabric module.

### Description of Related Art

Recent years, because people have paid more and more attention to their physical health, various types of detection devices that can help users detecting their own physiological conditions appear in the market.

For example, electrocardiogramachnology can monitor and record heart activities of users. However, when utilizing the electrocardiogramachnology, the users need to attach electrode patches to specific positions on bodies of the users for effective detection, which is quite inconvenient. For another example, some wearable devices (e.g., smart watches) can analyze and record physiological information (e.g., heart rate or blood oxygen) of the users by emitting laser light or light of a specific color to skin of the users and receiving reflected light. However, this type of wearable devices also needs to be worn on specific parts (e.g., a wrist) of users' body to measure effective physiological signals, which does not have flexibility for the users.

In summary, the market really needs to provide a novel technology that can be arbitrarily set at a non-fixed position to detect and analyze the physiological conditions of the users in a more flexible matter.

### SUMMARY OF THE DISCLOSURE

An object of the present disclosure is to provide a fabric module capable of detecting a physiological condition by utilizing a radio frequency signal and detection method for using the same, which can be set in a non-fixed position to detect and analyze the physiological conditions of the user in a more flexible matter.

In one of the exemplary embodiments, the fabric module capable of detecting a physiological condition by utilizing a radio frequency signal of the present disclosure includes:
a weaving body, configured for directly or indirectly contacting with a user;
a conductor group, attached to the weaving body, and having at least two conductors spaced apart from each other;
a signal processing unit, connected to one terminal of the at least two conductors, configured for emitting a radio frequency signal to the conductor group, and receiving a feedback signal generated by the conductor group based on the radio frequency signal; and
a sensing unit, connected to another terminal of the at least two conductors, so that the conductor group correspondingly generates the feedback signal based on the radio frequency signal;
where the signal processing unit is configured for generating a time domain periodic signal waveform diagram and a frequency domain periodic signal waveform diagram based on the feedback signal, and detecting a physiological condition of the user based on at least one of the time domain periodic signal waveform diagram and the frequency domain periodic signal waveform diagram.

In one of the exemplary embodiments, the physiological condition detection method of the present disclosure is applied to a fabric module which including a weaving body directly or indirectly contacting a user, a conductor group attached to the weaving body, a signal processing unit connected to one terminal of the conductor group, and a sensing unit connected to another terminal of the conductor group, where the physiological condition detection method includes:
step (a): by a transceiver modulation-demodulation unit of the signal processing unit, emitting a radio frequency signal to the conductor group through an impedance adapter, where the conductor group has at least two conductors spaced apart from each other;
step (b): by the transceiver modulation-demodulation unit, receiving a feedback signal generated by the conductor group based on the radio frequency signal;
step (c): by the signal processing unit, generating a time domain periodic signal waveform diagram and a frequency domain periodic signal waveform diagram based on the feedback signal; and
step (d): by the signal processing unit, detecting a physiological condition of the user based on at least one of the time domain periodic signal waveform diagram and the frequency domain periodic signal waveform diagram.

The present disclosure directly or indirectly contacts the user through the weaving body and the conductor group, so that the conductor group can be deformed by actions of the user, thereby correspondingly generating the feedback signal for the deformation. In this way, the present disclosure can detect the physiological condition of the user by the periodic signal waveform diagram generated based on the feedback signal for the deformation. Compared with related technology, the present disclosure does not need to restrict positions of the weaving body and the conductor group, and can record various types of physiological signals of the user based on deformation amounts and deformation frequencies of the conductor group, thereby more flexibly detecting the physiological condition of the user.

### BRIEF DESCRIPTION OF THE DRAWINGS

FIG. 1 is a schematic diagram of a fabric module according to a first embodiment of the present disclosure.

FIG. 2 is a block diagram of the fabric module according to the first embodiment of the present disclosure.

FIG. 3A is a schematic diagram of a conductor group according to the first embodiment of the present disclosure.

FIG. 3B is a schematic diagram of a deformation of the conductor group according to the first embodiment of the present disclosure.

FIG. 3C is a schematic diagram of a deformation of the conductor group according to a second embodiment of the present disclosure.

FIG. 4 is a schematic diagram of a time domain periodic signal waveform diagram according to the first embodiment of the present disclosure.

FIG. 5 is a schematic diagram of a frequency domain periodic signal waveform diagram according to the first embodiment of the present disclosure.

FIG. 6 is a schematic diagram of a time domain periodic signal waveform diagram according to the second embodiment of the present disclosure.

FIG. 7 is a flow chart of a detection method according to the first embodiment of the present disclosure.

FIG. 8 is a schematic diagram of a conductor group according to the second embodiment of the present disclosure.

### DETAILED DESCRIPTION

In cooperation with the attached drawings, the technical contents and detailed description of the present disclosure are described hereinafter according to multiple embodiments, being not used to limit its executing scope. Any equivalent variation and modification made according to appended claims is all covered by the claims claimed by the present disclosure.

The present disclosure discloses a fabric module capable of detecting a physiological condition by utilizing a radio frequency (RF) signal (hereinafter referred to as the fabric module). The fabric module can be utilized in combination with various daily necessities that can directly or indirectly contact with a user, such as clothes, a coat, a bed sheet, and a quilt, so as to detect the physiological condition of the user.

In one embodiment, the fabric module of the present disclosure can be a part of the daily necessities, for example, a weaving body of the fabric module can be directly utilized to make the bed sheet. In this way, when the user lies on a bed covered with the bed sheet, the fabric module can sense movements (e.g., turning over or getting out of the bed, etc.) of the user and thereby detect the physiological condition of the user.

In another embodiment, the fabric module of the present disclosure can be attached to the daily necessities, for example, the weaving body of the fabric module is sewn to the inside of the clothes. In this way, when the user wears the clothes sewn with the fabric module, the fabric module can sense movements (such as breathing) of the user and thereby detect the physiological condition of the user. However, the above descriptions are only some specific implementation examples of the present disclosure, but are not limited thereto.

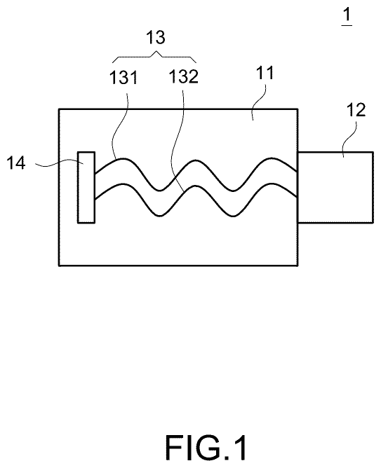

First, reference is made to FIG. 1, which is a schematic diagram of a fabric module 1 according to a first embodiment of the present disclosure. As shown in FIG. 1, the fabric module 1 of the present disclosure mainly includes a weaving body 11, a signal processing unit 12, a conductor group 13, and a sensing unit 14, where the signal processing unit 12 is connected to one terminal of the conductor group 13, and the sensing unit 14 is connected to another terminal of the conductor group 13.

In the embodiment of FIG. 1, the conductor group 13 is attached to the weaving body 11 and has at least two conductors (hereinafter referred to as a first conductor 131 and a second conductor 132) spaced apart from each other. The signal processing unit 12 is connected to one terminal of the first conductor 131 and the second conductor 132 to continuously emit a radio frequency signal to the conductor group 13 during detection and correspondingly receive a feedback signal generated by the conductor group 13 based on the radio frequency signal (described in more details below).

The sensing unit 14 is also attached to the weaving body 11 and is connected to another terminal of the first conductor 131 and the second conductor 132 relative to the signal processing unit 12. Specifically, the sensing unit 14 is a load with a specific impedance or an adjustable impedance. The first conductor 131 and the second conductor 132 are connected to each other through the sensing unit 14, so that the conductor group 13 can correspondingly generate the feedback signal based on the radio frequency signal being received.

In the present disclosure, the signal processing unit 12 is constituted by multiple internal components or modules (described more details below). In addition to emitting the radio frequency signal to the conductor group 13 and receiving the feedback signal from the conductor group 13, the signal processing unit 12 further selectively generates a time domain periodic signal waveform diagram and a frequency domain periodic signal waveform diagram based on the feedback signal. In this way, the signal processing unit 12 can detect the physiological condition of the user based on at least one of the time domain periodic signal waveform diagram and the frequency domain periodic signal waveform diagram.

As shown in FIG. 1, the first conductor 131 and the second conductor 132 in the present disclosure are not in a straight shape, but in the same bent shape, and the first conductor 131 and the second conductor 132 have a fixed spacing between each other. In one embodiment, the first conductor 131 and the second conductor 132 respectively include a copper wire and an insulation layer covering the copper wire. Through the arrangement of the insulation layer, it is possible to prevent the first conductor 131 and the second conductor 132 causing a short circuit due to being deformed by an external force.

In the present disclosure, the conductor group 13 is attached to the interior or surface of the weaving body 11, and the weaving body 11 is utilized to directly or indirectly contact the user wearing the fabric module 1. In other words, the conductor group 13 can also directly or indirectly contact the user. After receiving the radio frequency signal emitted by the signal processing unit 12, the first conductor 131 and the second conductor 132 generate respective capacitive reactance values (hereinafter referred to as a first capacitive reactance value and a second capacitive reactance value), and the first conductor 131 and the second conductor 132 jointly and correspondingly generate an inductance value. The feedback signal refers to a periodic signal waveform diagram generated based on the first capacitive reactance value, the second capacitive reactance value, and the inductance value.

The technical feature of the present disclosure is that the shape of the first conductor 131, the shape of the second conductor 132, and the spacing between the first conductor 131 and the second conductor 132 are all fixed and correspond to a preset state. The signal processing unit 12 continuously emits the radio frequency signal to the conductor group 13. If the shape of the first conductor 131, the shape of the second conductor 132, and the spacing between the first conductor 131 and the second conductor 132 do not change, the feedback signal generated by the conductor group 13 based on the radio frequency signal is fixed. When the user touches the fabric module 1, a force can be exerted on the fabric module 1 due to user's actions such as breathing, coughing, or turning over, thereby causing the first conductor 131 to deform, the second conductor 132 to deform, or the spacing between the first conductor 131 and the second conductor 132 to change. At this time, since at least one of the first capacitive reactance value, the second capacitive reactance value, and the inductance value have correspondingly changed, the feedback signal generated by the conductor group 13 based on the same radio frequency signal correspondingly changes as well. The technical feature of the present disclosure is that the signal processing unit 12 detects the physiological condition of the user in contact with the fabric module 1 based on changes in the feedback signal.

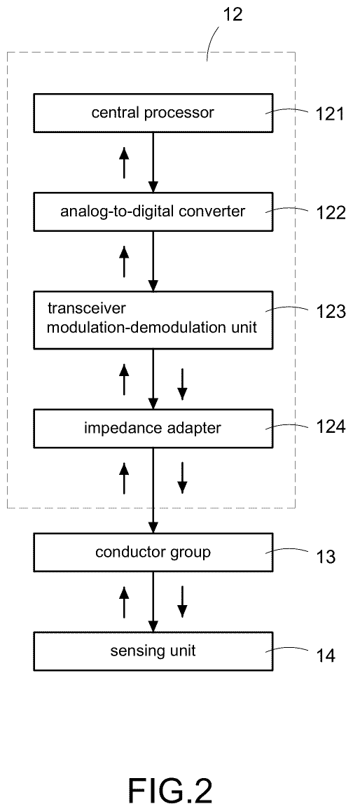

Reference is made to FIG. 2, which is a block diagram of the fabric module according to the first embodiment of the present disclosure. As shown in FIG. 2, the signal processing unit 12 of the present disclosure mainly includes a central processor 121, an analog-to-digital converter 122, a transceiver modulation-demodulation unit 123, and an impedance adapter 124, where the central processor 121 is connected to the analog-to-digital converter 122, the analog-to-digital converter 122 is connected to the transceiver modulation-demodulation unit 123, where the transceiver modulation-demodulation unit 123 is connected to the impedance adapter 124, and is connected to one terminal of the conductor group 13 (i.e., the first conductor 131 and the second conductor 132) through the impedance adapter 124.

As mentioned above, the transceiver modulation-demodulation unit 123 emits the radio frequency signal to the conductor group 13 through the impedance adapter 124, and receives the feedback signal generated by the conductor group 13 based on the radio frequency signal. The transceiver modulation-demodulation unit 123 executes demodulation processing on the feedback signal to generate a demodulated signal. In the present disclosure, the impedance adapter 124 is utilized to adjust the impedance to ensure that the radio frequency signal emitted by the transceiver modulation-demodulation unit 123 can be reliably emitted to the conductor group 13, so that the conductor group 13 can generate the feedback signal being required (e.g., the periodic signal waveform diagram).

In one embodiment, the transceiver modulation-demodulation unit 123 of the signal processing unit 12 continuously emits the radio frequency signal to the conductor group 13 based on a fixed frequency being preset, wherein the radio frequency signal is a sine wave signal. As mentioned above, when the conductor group 13 is deformed under the influence of the force made by the user, the feedback signal correspondingly changes. Therefore, a demodulation signal generated by the transceiver modulation-demodulation unit 123 is the feedback signal for a deformation (i.e., the demodulation signal of the deformation). More specifically, the demodulated signal generated by the transceiver modulation-demodulation unit 123 at least includes a voltage signal. In this way, the analog-to-digital converter 122 converts the demodulated signal being analog into a digital signal based on the voltage signal.

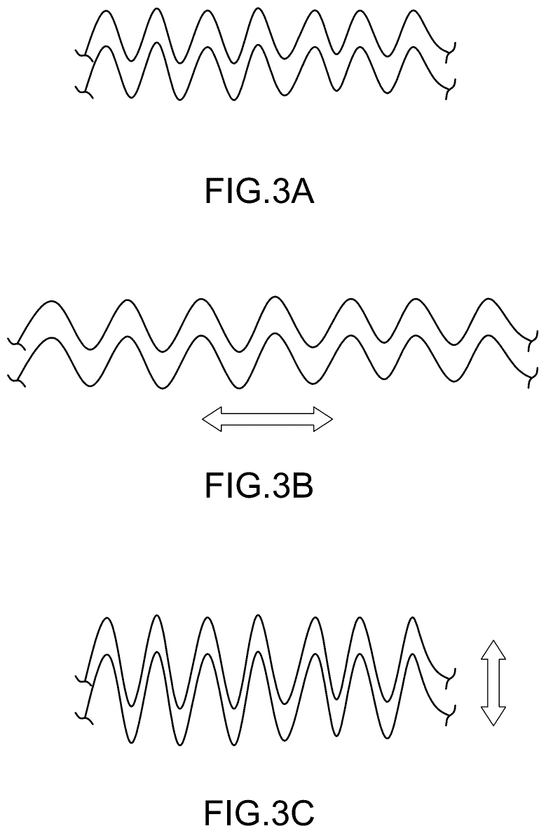

Reference is made to FIGS. 3A-3C, which are respectively a schematic diagram of a conductor group according to the first embodiment of the present disclosure, a schematic diagram of a deformation of the conductor group according to the first embodiment of the present disclosure, and a schematic diagram of a deformation of the conductor group according to a second embodiment of the present disclosure.

FIG. 3A discloses the schematic diagram of the conductor group 13 of the present disclosure. It can be seen from FIG. 3A that the first conductor 131 and the second conductor 132 have the same bent shape and have the fixed spacing between each other. In the embodiment of FIG. 3B, the conductor group 13 is stretched in a horizontal direction under the influence of the user (e.g., the fabric module 1 is sewn on the clothes worn by the user and user's breathing causes the conductor group 13 to stretch in the horizontal direction). At this time, because the first capacitive reactance value generated by the first conductor 131 based on the radio frequency signal, the second capacitive reactance value generated by the second conductor 132 based on the radio frequency signal, and the inductance value generated by the first conductor 131 and the second conductor 132 all correspondingly change, the feedback signal received by the transceiver modulation-demodulation unit 123 is the feedback signal for the deformation, and the demodulated signal generated after demodulation is the demodulated signal of the deformation.

In the embodiment of FIG. 3C, the conductor group 13 is stretched in a vertical direction under the influence of the user (e.g., the fabric module 1 is woven in the bed sheet and the conductor group 13 is stretched in the vertical direction due to the pressure of the user standing up). At this time, because the first capacitive reactance value generated by the first conductor 131 based on the radio frequency signal, the second capacitive reactance value generated by the second conductor 132 based on the radio frequency signal, and the inductance value generated by the first conductor 131 and the second conductor 132 all correspondingly change, the feedback signal received by the transceiver modulation-demodulation unit 123 is the feedback signal for the deformation, and the demodulated signal generated after demodulation is the demodulated signal of the deformation.

Return to FIG. 2. The analog-to-digital converter 122 receives the demodulation signal generated by the transceiver modulation-demodulation unit 123 from the transceiver modulation-demodulation unit 123, and executes analog-to-digital conversion processing on the demodulated signal to correspondingly generate the digital signal. The analog-to-digital conversion processing is a common knowledge in the field of signal processing technology, and will not be described in detail here.

The central processor 121 receives the converted digital signal from the analog-to-digital converter 122, and executes signal conversion processing on the digital signal to generate the time domain periodic signal waveform diagram and the frequency domain periodic signal waveform diagram. Furthermore, the central processor 121 can detect the physiological condition of the user in contact with the fabric module 1 based on at least one of the time domain periodic signal waveform diagram and the frequency domain periodic signal waveform diagram. The signal conversion processing can be, for example, fast Fourier transform processing, but is not limited thereto. In the present disclosure, the central processor 121 determines whether the user has an emergency event (e.g., falling, turning over, or getting out of the bed, etc.) through the time domain periodic signal waveform diagram that brings time information and instantaneous voltage change information, and detects continuous physiological information (e.g., breathing or heartbeat, etc.) of the user through the frequency domain periodic signal waveform diagram that brings frequency information and signal strength information.

For example, after the central processor 121 generates the time-domain periodic signal waveform diagram, at least one surge shape greater than a preset threshold may be found in the time domain periodic signal waveform diagram. Since the surge shape in the waveform diagram represents an instantaneous change (e.g., an instantaneous action of the user causes the conductor group 13 to instantaneously deform), the central processor 121 utilizes this to determine that the emergency event has occurred for the user. For another example, after the central processor 121 generates the frequency domain periodic signal waveform diagram, the central processor 121 continuously observes the signal strength and frequency in the frequency domain periodic signal waveform diagram, and records the signal strength and frequency as the continuous physiological information such as the breathing or heartbeat of the user. In this way, the central processor 121 determines whether the physiological condition of the user such as the breathing or heartbeat are normal.

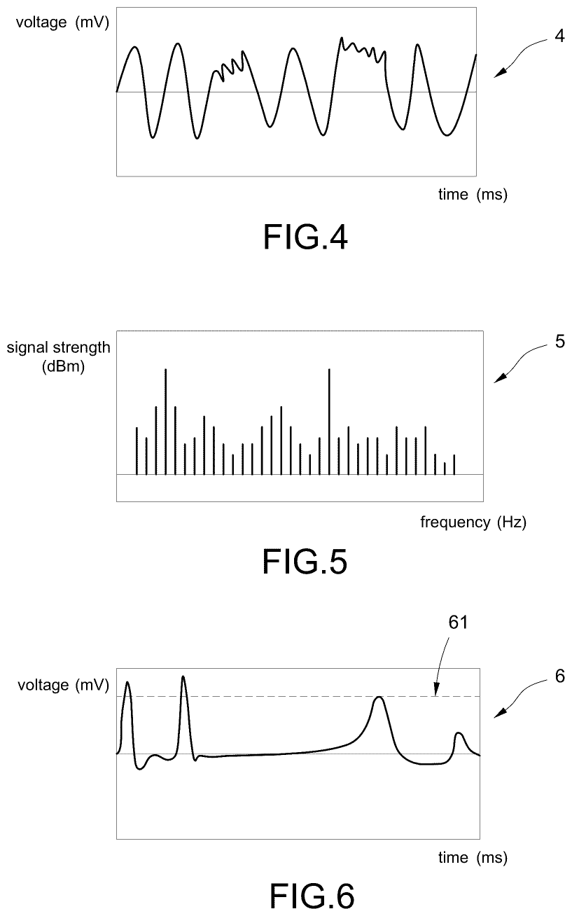

Reference is made to FIG. 4, FIG. 5, and FIG. 6 at the same time, which respectively are a schematic diagram of a time domain periodic signal waveform diagram according to the first embodiment of the present disclosure, a schematic diagram of a frequency domain periodic signal waveform diagram according to the first embodiment of the present disclosure, and a schematic diagram of a time domain periodic signal waveform diagram according to the second embodiment of the present disclosure. FIG. 4 discloses the time domain periodic signal waveform diagram 4 generated by the central processor 121 based on the feedback signal of the conductor group 13. In the embodiment of FIG. 4, the change of the waveform over time represents the deformation of the conductor group 13. For example, when the user normally breathes, his breathing action causes continuous and regular deformations of the conductor group 13, and a breathing frequency affects the shape of the waveform in the time domain periodic signal waveform in FIG. 4.

In the embodiment of FIG. 4, a special surge does not exist in the time domain periodic signal waveform diagram 4, so the central processor 121 further generates the frequency domain periodic signal waveform diagram 5 shown in FIG. 5. In this way, the central processor 121 can detect and record the continuous physiological information (e.g., the breathing or heartbeat) of the user based on the signal strength and frequency in the frequency domain periodic signal waveform diagram 5. In the embodiment of FIG. 6, the central processor 121 observes the at least one surge shape greater than the preset threshold 61 in the time domain periodic signal waveform diagram 6, where this surge shape represents instantaneous violent deformation of the conductor group 13. For example, when the user is lying on the bed sheet with the fabric module 1 sewn on and suddenly gets out of bed, his movement of getting out of the bed causes the conductor group 13 to instantaneously deform and makes the surge shape appear in the time domain periodic signal waveform diagram in FIG. 6.

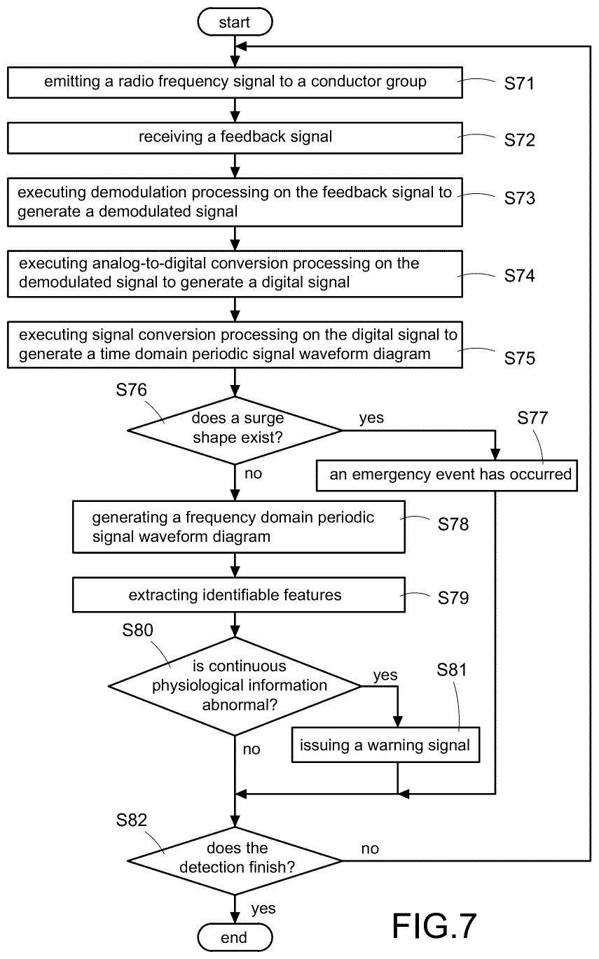

Reference is made to FIG. 7, which is a flow chart of a detection method according to the first embodiment of the present disclosure. FIG. 7 discloses specific implementation steps of the detection method of the present disclosure, and this detection method is applied to the fabric module 1 shown in FIGS. 1 and 2.

The weaving body 11 of the present disclosure can be, for example, various elastic fabrics, and be applied to the daily necessities of the user, such as the clothes or the bed sheet. The conductor group 13 is arranged in the interior or on the surface of the weaving body 11. The conductors 131 and 132 are spaced apart from each other, and the insulation layer on the surface of the conductors 131 and 132 prevents each other from causing short circuit. When the user uses the daily necessity with the fabric module 1, the fabric module 1 emits the radio frequency signal to the conductor group 13 through the impedance adapter 124 by the transceiver modulation-demodulation unit 123 in the signal processing unit 12 (step S71), and receives the feedback signal generated by the conductor group 13 based on the radio frequency signal by the transceiver modulation-demodulation unit 123 (step S72) too.

In step S71, the transceiver modulation-demodulation unit 123 mainly emits the sine wave signal to the conductor group 13 based on the fixed frequency being preset. Each conductor (such as the first conductor 131 and the second conductor 132) in the conductor group 13 respectively generates its own capacitive reactance value after receiving the radio frequency signal, and jointly and correspondingly generates the inductance value. The capacitance values and the inductance value constitute the feedback signal. Therefore, during the processing of the steps S71 and S72, if the conductor group 13 is deformed by the external force of the user, the transceiver modulation-demodulation unit 123 receives the feedback signal for the deformation.

After step S72, the signal processing unit 12 can correspondingly generate the time domain periodic signal waveform diagram and the frequency domain periodic signal waveform diagram based on the feedback signal, and then detect the physiological condition (e.g., a breathing condition, a heartbeat condition, whether the user fells, whether the user turns over or gets out of the bed, etc., but are not limited thereto) of the user who uses the fabric module 1 based on at least one of the time domain periodic signal waveform diagram and the frequency domain periodic signal waveform diagram.

Specifically, after step S72, the signal processing unit 12 executes the demodulation processing on the feedback signal received through the transceiver modulation-demodulation unit 123 to generate the demodulated signal (step S73). Next, the signal processing unit 12 executes the analog-to-digital conversion processing on the demodulated signal by the analog-to-digital converter 122 to correspondingly generate the digital signal (step S74). Next, the signal processing unit 12 executes the signal conversion processing on the digital signal by the central processor 121, thereby generating the time domain periodic signal waveform diagram utilized for subsequent detection (step S75). In one embodiment, the central processor 121 simultaneously generates the time domain periodic signal waveform diagram and the frequency domain periodic signal waveform diagram by executing the signal conversion processing in step S75. In another embodiment, the central processor 121 only generates the time domain periodic signal waveform diagram in step S75, and only generates the frequency domain periodic signal waveform diagram when no special signal (e.g., the surge shape greater than the preset threshold) exists in the time domain periodic signal waveform.

After step S75, the central processor 121 observes whether the at least one surge shape greater than the preset threshold exists in the time domain periodic signal waveform diagram (step S76). If the at least one surge shape greater than the preset threshold exists in the time domain periodic signal waveform diagram, the central processor 121 determines that the emergency event has occurred to the user (step S77). Specifically, the surge shape represents the instantaneous change, so the appearance of the surge shape greater than the preset threshold in the time domain periodic signal waveform diagram represents that the user has performed the instantaneous action where this instantaneous action is regarded as the emergency event. It should be noted that a manufacturer of the fabric module 1 tests possible actions of the user and a deformation amount for the conductor group 13 due to the position (e.g., the clothes, the pants, or the bed sheets, etc.) where the fabric module 1 is disposed, thereby setting the preset threshold and a quantity of the surge shape. In this way, in step S77, the central processor 121 identifies the emergency event (such as the falling, the turning over, or the getting out of the bed, etc., but are not limited thereto) for the user based on the size and quantity of the surge shape according to a preset rule.

If in step S75, no surge shape that is greater than the preset threshold or much more than the preset quantity is observed in the time domain periodic signal waveform diagram, the central processor 121 further executes the signal conversion processing on the digital signal or the time domain periodic signal waveform diagram to generate the frequency domain periodic signal waveform diagram (step S78). However, as mentioned above, the central processor 121 can generate the time domain periodic signal waveform diagram and the frequency domain periodic signal waveform diagram simultaneously in step S75, and is not limited to the step flow shown in FIG. 7.

Next, the central processor 121 extracts identifiable features from the frequency domain periodic signal waveform diagram (step S79). In one embodiment, the identifiable features are the signal strength and frequency. In one embodiment, the signal strength corresponds to the deformation amount of the conductor group 13 (e.g., the force of the breathing of the user), and the frequency corresponds to a deformation frequency of the conductor group 13 (such as the frequency of the user's breathing). That is, the signal strength combined with the frequency represent a continuous dynamic action of the user. In this way, the central processor 121 determines whether the continuous physiological information of the user such as the breathing or heartbeat is abnormal based on the signal strength and frequency in the frequency domain periodic signal waveform diagram (step S80), and issues a warning signal when the abnormality has determined (Step S81).

During the execution processing of the detection method of the present disclosure, the central processor 121 continuously determines whether stopping the detection (step S82). For example, the central processor 121 determines stopping the detection when the fabric module 1 is powered off. Before the detection needs to be stopped, the fabric module 1 continuously executes steps S71 to S82 to continuously generate the time domain periodic signal waveform diagram and the frequency domain periodic signal waveform diagram according to the movement of the user on the fabric module 1, thereby continuously detecting the physiological condition of the user.

In the aforementioned embodiments, a quantity of the conductors in conductor group 13 is two as an example. However, in other embodiments, the quantity of the conductors can be any positive integer greater than two, where any two adjacent conductors of the multiple conductors are spaced apart from each other with a fixed and equal spacing. For example, if the quantity of the conductors in the conductor group 13 is three (including a first conductor, a second conductor, and a third conductor), the first conductor, the second conductor, and the third conductor exhibit the same bent shape, the first conductor is adjacent (with a first spacing) to the second conductor, the second conductor is adjacent (with a second spacing) to the third conductor, and the first spacing is equal to the second spacing. After receiving the radio frequency signal, the first conductor correspondingly generates a first capacitive reactance value, the second conductor correspondingly generates a second capacitive reactance value, the third conductor correspondingly generates a third capacitive reactance value, the first conductor and the second conductor can jointly generate a first inductance value, and the second conductor and the third conductor can jointly generate a second inductance value. In this embodiment, the feedback signal of the conductor group 13 is generated by the first capacitive reactance value, the second capacitive reactance value, the third capacitive reactance value, the first inductance value, and the second inductance value.

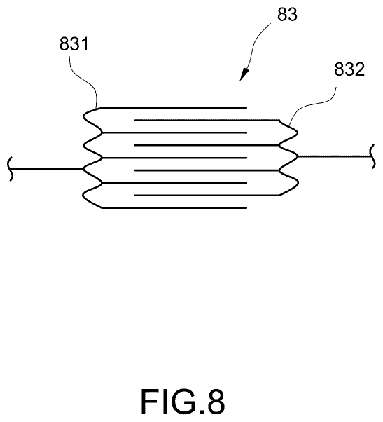

In the aforementioned embodiments, the conductors in the conductor group 13 are in the bent shape as an example, but it is not limited thereto. Reference is made to FIG. 8, which is a schematic diagram of a conductor group according to the second embodiment of the present disclosure. In the embodiment of FIG. 8, the conductor group 83 includes a first conductor 831 and a second conductor 832. One terminal of the first conductor 831 and the second conductor 832 exhibits one or more tuning fork shapes, and these tuning fork shapes are arranged crosswise with the same spacing. In this embodiment, the technical method by which the conductor group 83 correspondingly generates a feedback signal based on the radio frequency signal is the same as that of the aforementioned conductor group 13, and will not be described again.

The present disclosure makes the fabric module sew in an unfixed position, and detects the deformation (including the deformation amount and the deformation frequency) caused by the force of the user on the conductor group, thereby more flexibly analyzing the physiological condition of the user.

As the skilled person will appreciate, various changes and modifications can be made to the described embodiment. It is intended to include all such variations, modifications and equivalents which fall within the scope of the present disclosure, as defined in the accompanying claims.

What is claimed is:

1. A fabric module capable of detecting a physiological condition by utilizing a radio frequency signal, comprising:
a weaving body, configured for directly or indirectly contacting with a user;
a conductor group, attached to the weaving body, and having at least two conductors spaced apart from each other;
a signal processing unit, connected to one terminal of the at least two conductors, configured for emitting a radio frequency signal to the conductor group, and receiving a feedback signal generated by the conductor group based on the radio frequency signal; and
a sensing unit, connected to another terminal of the at least two conductors, so that the conductor group correspondingly generates the feedback signal based on the radio frequency signal;
wherein the signal processing unit is configured for generating a time domain periodic signal waveform diagram and a frequency domain periodic signal waveform diagram based on the feedback signal, and detecting a physiological condition of the user based on at least one of the time domain periodic signal waveform diagram and the frequency domain periodic signal waveform diagram.

2. The fabric module capable of detecting the physiological condition by utilizing the radio frequency signal in claim 1, wherein the at least two conductors respectively comprise a copper wire and an insulation layer covering the copper wire, the at least two conductors exhibit same bent shape, and a fixed spacing exists between the at least two conductors.

3. The fabric module capable of detecting the physiological condition by utilizing the radio frequency signal in claim 1, wherein the at least two conductors respectively generate respective capacitive reactance values after receiving the radio frequency signal, and the at least two conductors jointly generate an inductance value after receiving the radio frequency signal, wherein the feedback signal is a periodic signal waveform diagram generated based on the two capacitive reactance values and the inductance value, and when the at least two conductors receive an external force to deform, the at least two capacitive reactance values, the inductance value, and the feedback signal correspondingly change.

4. The fabric module capable of detecting the physiological condition by utilizing the radio frequency signal in claim 1, wherein the signal processing unit is configured for emitting the radio frequency signal based on a fixed frequency being preset, and the radio frequency signal is a sine wave signal.

5. The fabric module capable of detecting the physiological condition by utilizing the radio frequency signal in claim 1, wherein the signal processing unit comprises:
an impedance adapter, connected to the conductor group;
a transceiver modulation-demodulation unit, connected to the impedance adapter, configured for emitting the radio frequency signal to the conductor group through the impedance adapter, and executing a demodulation processing on the feedback signal to generate a demodulation signal;
an analog-to-digital converter, connected to the transceiver modulation-demodulation unit, and configured for executing an analog-to-digital conversion processing on the demodulated signal to generate a digital signal; and
a central processor, connected to the analog-to-digital converter, and configured for executing a signal conversion processing on the digital signal to generate the time domain periodic signal waveform diagram and the frequency domain periodic signal waveform diagram, and detecting the physiological condition of the user based on at least one of the time domain periodic signal waveform diagram and the frequency domain periodic signal waveform diagram.

6. The fabric module capable of detecting the physiological condition by utilizing the radio frequency signal in claim 5, wherein the central processor is configured for determining that an emergency event occurs to the user when at least one surge shape greater than a preset threshold exists in the time domain periodic signal waveform diagram, and determining whether the user's breathing or heartbeat is normal based on a signal strength and a frequency in the frequency domain periodic signal waveform diagram.

7. A physiological condition detection method of a fabric module, applied to the fabric module comprising a weaving body directly or indirectly contacting a user, a conductor group attached to the weaving body, a signal processing unit connected to one terminal of the conductor group, and a sensing unit connected to another terminal of the conductor group, and the physiological condition detection method comprising:
step (a): by a transceiver modulation-demodulation unit of the signal processing unit, emitting a radio frequency signal to the conductor group through an impedance adapter, wherein the conductor group comprises at least two conductors spaced apart from each other;
step (b): by the transceiver modulation-demodulation unit, receiving a feedback signal generated by the conductor group based on the radio frequency signal;
step (c): by the signal processing unit, generating a time domain periodic signal waveform diagram and a frequency domain periodic signal waveform diagram based on the feedback signal; and
step (d): by the signal processing unit, detecting a physiological condition of the user based on at least one of the time domain periodic signal waveform diagram and the frequency domain periodic signal waveform diagram.

8. The physiological condition detection method in claim 7, wherein the at least two conductors respectively comprise a copper wire and an insulation layer covering the copper wire, the at least two conductors exhibit same bent shape, and a fixed spacing exists between the at least two conductors.

9. The physiological condition detection method in claim 7, wherein the step (b) comprises:
step (b1): by the at least two conductors, respectively generating respective capacitive reactance values after receiving the radio frequency signal; and
step (b2): by the at least two conductors, jointly generating an inductance value after receiving the radio frequency signal;
wherein the feedback signal is a periodic signal waveform generated based on the at least two capacitive reactance values and the inductance value;
wherein when the at least two conductors receive an external force to deform, the at least two capacitive reactance values, the inductance value, and the feedback signal correspondingly change.

10. The physiological condition detection method in claim 7, wherein the step (a) comprises:
by the transceiver modulation-demodulation unit, emitting the radio frequency signal based on a fixed frequency being preset, wherein the radio frequency signal is a sine wave signal.

11. The physiological condition detection method in claim 7, wherein the step (c) comprises:
step (c1): by the transceiver modulation-demodulation unit, executing a demodulation processing on the feedback signal to generate a demodulated signal;
step (c2): by an analog-to-digital converter of the signal processing unit, executing an analog-to-digital conversion processing on the demodulated signal to generate a digital signal;
step (c3): by a central processor of the signal processing unit, executing a signal conversion processing on the digital signal to generate the time domain periodic signal waveform diagram; and
step (c4): when no surge shape that is greater than a preset threshold exists in the time domain periodic signal waveform diagram, generating, by the signal processing unit, the frequency domain periodic signal waveform diagram based on the digital signal.

12. The physiological condition detection method in claim 11, wherein the step (d) comprises:
step (d1): when at least one surge shape greater than the preset threshold exists in the time domain periodic signal waveform diagram, determining, by the central processor, that an emergency event occurs to the user; and
step (d2): by the central processor, determining whether the user's breathing or heartbeat is normal based on a signal strength and a frequency in the frequency domain periodic signal waveform diagram.
